(19) United States
(12) Patent Application Publication
(10) Pub. No.: US 2025/0058062 A1
(43) Pub. Date: Feb. 20, 2025

(54) Device and method for dispensing volatile substances

(71) Applicant: CTR, LDA, Samora Correia (PT)

(72) Inventors: Harold Augier, Zhongshan Guangdong (CN); Luo Min, Zhongsham City (CN)

(21) Appl. No.: 18/722,966

(22) PCT Filed: Dec. 23, 2021
(86) PCT No.: PCT/EP2021/087469

§ 371 Date: Jun. 21, 2024

(51) Int. Cl. A61M 11/04

(52) U.S. Cl. CPC A61M 11/042; A61M 2205/3368; A61M 2205/8262

(57) ABSTRACT

The invention relates to a device for dispensing volatile substances, having a housing (2), a container (5) for the substance to be dispensed, a wick received in the container (5) and protruding from the container (5) with a wick end (6) forming a substance dispensing area, and further having an electrical heating device (8) which has an electrical heating circuit, the heating device (8) being displaceable relative to the wick end (6) in order to set a substance delivery rate. According to the invention the electrical heating circuit of the heating device (8) comprises a thermoswitch (16) being openable by heat exposure at a predetermined switching temperature and being arranged at a distance from the wick end (6), the thermoswitch (16) being switchable as a function of the distance between the heating device (8) and the thermoswitch (16).

The invention relates to a device for dispensing volatile substances, in particular fragrances and/or active ingredients, according to the preamble of claim 1. Furthermore, the invention relates to a method for operating a device for dispensing volatile substances, in particular fragrances and/or active ingredients, according to the preamble of claim 13.

A known device for vaporizing volatile substances, in particular fragrances and/or active substances (EP 1 108 358 B1) comprises a housing and a container in which a substance to be dispensed is accommodated and which is at least partially insertable into the housing and connectable to the housing. A wick is arranged in the container as a capillary element, which projects beyond the container with a free wick end and is in contact with the substance to be dispensed in such a way that the substance is conveyed towards the free wick end by means of the capillary action of the wick. In addition, an electrical heating device with at least one electrical heating element is arranged in the housing, which is associated with the free wick end and by means of which heat can be applied to the free wick end in order to be able to rapidly release the substance accumulating in the free wick end into the environment.

The heating device can be displaced and fixed in different positions in the housing relative to the wick end in order to set a substance delivery rate, i.e. a release rate for the substance to be released, in such a way that in positions near or close to the wick (hereinafter referred to as near-wick positions) there is a high heat transfer between the heating device and the wick end with a high substance delivery rate and in positions remote from the wick (hereinafter referred to as remote-wick positions) a comparatively lower substance delivery rate is set with a correspondingly lower heat transfer. In this case, the heating device is displaceably mounted on a linear sliding guide.

For the strongest possible effect, the user of the device very often sets the highest substance delivery rate with the smallest distance between the heating device and the wick end. This can have the disadvantage that the free wick end dries out and dehumidifies strongly due to a very high wick temperature, whereby the capillary structure in the wick end can be damaged by sticking and/or caking together to such an extent that the substance delivery rate is considerably reduced and, in extreme cases, no more substance evaporates and a partially still filled container has to be replaced.

On the other hand, if the heating device is set in the other extreme position with maximum distance to the end of the wick, the substance delivery rate may be so low that, although this avoids damage to the wick, a desired effect can only be achieved insufficiently. If necessary, a user can try to find an intermediate position of the heating device for a compromise between the two disadvantages explained above by carrying out his own tests, but this requires time-consuming adjustments and is not usually done.

It is therefore an object of the invention to create a device which has a simple and inexpensive structure, is further easy to handle and provides the consumer with a simple means of individually setting the substance delivery rate. A further object of the invention is to propose a suitable method for operating such a device.

This object of the invention is solved with respect to the device by means of the features of claim 1. Advantageous embodiments thereof are the subject of the subclaims referring back thereto.

According to claim 1, a device for dispensing, in particular for vaporizing, volatile substances, for example fragrances and/or active ingredients, is provided, comprising a housing and a container for the substance to be dispensed. According to a preferred embodiment, the container is connectable to the housing, preferably detachably connectable, and/or at least partially insertable into the housing.

Further, a wick is provided having a portion received in the container and further extending from the container with a wick end forming a substance dispensing portion.

In addition, an electrical heating device is provided, preferably arranged in the housing, which comprises an electrical heating circuit. The electrical heating device preferably comprises at least one electrical heating element which forms part of the electrical heating circuit.

The heating device is displaceable relative to the wick end when the container is inserted and/or for setting a substance delivery rate.

According to the invention, the electrical heating circuit of the heating device comprises a thermoswitch respectively thermal switch being openable by heat exposure at a predetermined switching temperature and being arranged at a distance from the wick end, the thermoswitch being switchable as a function of the distance between the heating device and the thermoswitch, for example being open, remaining open or becoming open or being closed, remaining closed or becoming closed.

This advantageously achieves that the electrical heating circuit of the heating device is interrupted in the open state of the thermoswitch or that the heating device is switched off, in particular temporarily switched off, in the open state of the thermoswitch. This results in advantageous individual setting options for the user, which is in particular also illustrated once again by the following description of preferred embodiments.

It is understood that the thermoswitch, even if this is not explicitly mentioned each time, can open by heat exposure at a predetermined switching temperature and can (re) close when the temperature cools below the switching temperature, possibly with a hysteresis. The thermoswitch used in accordance with the invention is also preferably designed to detect a temperature and, depending on the detected temperature, to assume a preset, specific switching state. The thermoswitch can be designed as a thermistor, for example as a PTC resistor (Positive Temperature Coefficient resistor), or particularly preferably as a bimetal switch.

According to a particularly preferred specific embodiment, it is provided that the heating device for setting a substance delivery rate is displaceable relative to the wick end into a near-wick position and into a remote-wick position, with the heating device being arranged closer to the wick end in the near-wick position than in the remote-wick position and with a greater amount of heat being transferred to the wick end by the heating device, in its activated state, in the near- wick position than in the remote-wick position and, accordingly, the substance delivery rate is greater in the near-wick position than in the remote-wick position. Moreover, it is provided that the distance of the heating device from the thermoswitch is greater in the near-wick position than in the remote-wick position. Accordingly, the distance of the heating device from the thermoswitch in the near-wick position corresponds to a position remote from the switch and the distance of the heating device from the thermoswitch in the remote-wick position corresponds to a position near or close to the switch.

Furthermore, in this specific embodiment, it is provided that the thermoswitch is switchable depending on the distance between the heating device and the thermoswitch in such way, and that the heating power of the heating device and the switching temperature of the thermoswitch are set in such way:
a) That in the near-wick position (corresponding to the position remote from the switch) of the heating device, the heat transfer to the thermoswitch is too low to reach the switching temperature and accordingly the thermoswitch closes or remains closed. As a result, the device can be operated continuously at high wick temperature and high substance delivery rate in this near-wick position because the thermoswitch is constantly closed in this near-wick position. A user achieves a large effect with high substance delivery rate with this setting, but possibly with a risk that the capillary structure of the wick end may be damaged after a longer operation of the device. However, if the device is switched to the remote-wick position soon, this risk is low and controllable.
b) That in the remote-wick position (corresponding to the position near or close to the switch) of the heating device the heat transfer to the thermoswitch is sufficient to reach the switching temperature and accordingly the thermoswitch opens to switch off the electrical heating device. Preferably, this is done in such a way that the thermoswitch repeatedly switches on and off for alternating operation of the device in this remote-wick position of the heating device, in particular in such a way that the thermoswitch switches off (opens) in each case after a heating time and switches on (closes) again after a cooling time. In this way, a wick-protecting, substance-consuming and long-lasting operation of the device can be achieved with a sufficient substance delivery rate. In addition, the wick temperature here then preferably oscillates around an average wick temperature between a between a basically achievable maximum and minimum temperature.

As an alternative to the embodiment according to the above points a) and b), it can be provided that the thermoswitch is switchable depending on the distance between the heating device and the thermoswitch in such way, and that the heating power of the heating device and the switching temperature of the thermoswitch are set in such way, that both in the remote-wick position of the heating device and in the near-wick position of the heating device the heat transfer to the thermoswitch is sufficient to reach the switching temperature and accordingly the thermoswitch opens upon reaching the switching temperature for switching off the electrical heating device such that the thermoswitch repeatedly switches on and off both in the remote-wick position of the heating device and in the near wick position of the heating device for an alternating operation of the device, although with the difference that the switch-on and switch-off cycles in the remote-wick position are different, in particular shorter, than in the near wick position. This provides different switch-on and switch-off cycles which can also cause a switch-off in the near-wick position, which means that a user can still achieve a large effect with a high substance delivery rate in a near-wick position, but with a lower risk of damage to the capillary structure of the wick end compared to continuous operation. Due to the greater distance of the heating device from the thermoswitch in the near-wick position, it thus takes longer for the thermoswitch to reach the switching temperature, especially in relation to a comparable identical initial or reference state (i.e., for example, with reference to heating up the heating device in the near-wick and remote-wick positions, each starting from the room temperature), so that the time required to open the thermoswitch and thus to switch off the heating device is correspondingly longer in the near-wick position than in the remote-wick position. As a result, the heating phase of the heating device in the near-wick position is generally longer and, accordingly, the heating device generally reaches higher heating temperatures than in the far-wick position. In this respect, it generally also takes longer for the heating device in the near-wick position to cool down to the switching temperature, so that accordingly the cooling phase in the near-wick position also generally takes longer than in the far-wick position. In line with this assumption, the switch-on and switch-off cycles in the remote-wick position are shorter than in the near-wick position.

According to a particularly preferred embodiment, which is simple in terms of design and can be controlled in a functionally reliable manner in conjunction with the provision of a thermoswitch, it is provided that the heating device can be displaced exclusively between the remote-wick position and the near-wick position and, in this respect, only two setting positions are provided. With regard to the different setting positions, however, the following applies in principle: It is true that the idea of the invention is described and explained in more detail herein in particular with reference to the preferred setting positions designated as the near-wick position and the remote-wick position. However, this expressly does not mean that a device designed in accordance with the present invention must always have only these two setting positions, although this can of course be the case, as already explained above, because it is a particularly preferred embodiment in this respect. Rather, further setting positions, for example intermediate positions lying between these two positions, can of course also be set and/or preset, in which, for example, the effect of interaction with the thermoswitch brought about or desired in connection with the remote-wick position or the near-wick position is more or less given or, as the case may be, is not given at all or is given only in a modified form. These embodiments also expressly do not represent a workaround solution, but are to be expressly regarded as encompassed by the present inventive idea.

The thermoswitch can basically be arranged at any suitable position in or on the housing. A protected arrangement of the thermoswitch in the interior of the housing is particularly functionally reliable.

The thermoswitch is also preferably arranged in a fixed position. This means that a fixed distance to the wick end is then predetermined and set, and the device can be configured and manufactured correspondingly simply. In principle, however, an embodiment is also possible in which the thermoswitch is displaceable, e.g. in order to be able to variably set the distance to the wick end.

In a specific first embodiment, the heating device comprises a single electrical heating element switchable by the thermoswitch. This electrical heating element is preferably formed by a heating resistor. In such an embodiment, the wick temperature in the remote-wick position then preferably oscillates around an average temperature value which is, for example, in the range of 70° to 90° C. In a practical embodiment, for example, this average temperature value is about 80° C. and thus approximately midway between an upper, maximum temperature value of about 90° C. and a lower, minimum temperature value of about 70° C.

In a second specific embodiment alternative thereto, the heating device may have two electrical heating elements arranged in parallel, in particular heating resistors as electrical heating elements, one of the two heating elements being switchable with the thermoswitch, while the other heating element, on the other hand, heats continuously. Here, too, the wick temperature oscillates about a mean value, whereby, in comparison with the above first embodiment with a single heating element, the cooling times can be longer and the temperatures can be dimensioned larger, for example in such a way that the wick temperature in the remote-wick position also oscillates, as in the first embodiment, about a mean temperature value, preferably about 80° C., between an upper temperature value of then, for example, about 100° C. and a lower temperature value of then, for example, about 60° C.

In a particularly preferred embodiment in connection with the present invention, the heating device can have a heating body, preferably plate-shaped and/or cuboid-shaped and/or annular, with a wick-receiving opening, preferably formed through-going, for the wick end, or can be formed by such a heating body. With such a heating device, its outer contour is clearly defined, so that it is possible to orientate oneself very well on it for the constructive arrangement and design of the remaining components, in particular of the thermoswitch. The wick receiving opening can be designed in different ways, e.g. as a central or middle wick receiving opening or as a wick recess at the edge, to name just a few specific examples.

It can further be provided, for example, that an electrical heating element, preferably an electrical resistor as an electrical heating element, is embedded in the heating body, which is produced at least in regions, preferably predominantly, most preferably completely, from a thermally conductive material, in particular a thermally conductive ceramic or plastic material, and is a component of the electrical heating circuit. Such a heating body can be manufactured simply, quickly and inexpensively.

In principle, the displacement or, for example, height adjustment of the heating device can be effected in different ways, for example by a rotary adjustment in which the heating device is rotated and displaced in a screw-like manner into different setting positions. According to a particularly preferred embodiment, however, it is provided that the heating device is linearly displaceable on or along a sliding guide. Such a linear sliding guide enables a simple and fast as well as, above all, functionally reliable adjustment between the respectively provided setting positions, in particular the preferred setting positions of a remote-wick position and a near-wick position. Particularly preferred in this context is a specific embodiment in which the wick end projects into the wick-receiving opening in the near-wick position and in which the wick-receiving opening lies, relative to the wick vertical axis direction, at a distance above the wick end in the remote-wick position and accordingly has a smaller distance from the thermoswitch than in the near-wick position.

The device may further preferably be in the form of a plug component having plug contacts projecting from the housing, the plug contacts being insertable into an electrical socket for supplying electrical power and for holding the housing in place.

The object of the invention is solved with respect to the method with the features of claim 12. Advantageous embodiments thereof are the subject of the subclaims referring back thereto.

According to claim 12, there is provided a method of operating a device for dispensing volatile substances, the device comprising a housing, a container for the substance to be dispensed, a wick received in the container and protruding from the container with a wick end forming a substance dispensing region, and further comprising an electrical heating device which in turn comprises an electrical heating circuit, the heating device being displaceable relative to the wick end to adjust a substance delivery rate. According to the invention, it is provided that the electrical heating circuit of the heating device comprises a thermoswitch which is openable by heat exposure at a predetermined switching temperature and which is arranged at a distance from the wick end, the thermoswitch being switched as a function of the distance between the heating device and the thermoswitch.

The advantages resulting therefrom are identical to those of the device according to the invention. In this respect, in order to avoid repetition, reference is made to the advantages of the device mentioned above.

With reference to the below drawings, the invention will be further explained by way of examplary embodiments:

FIG. 1a shows a perspective view of a device for dispensing volatile substances as a connector component;

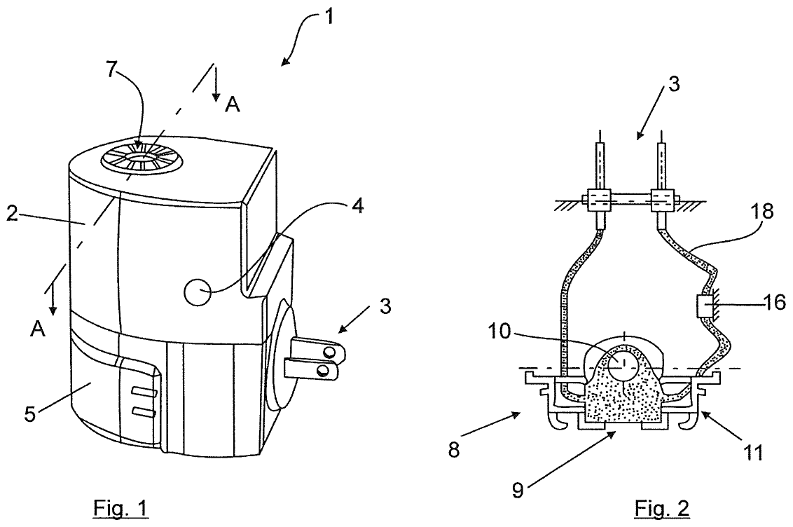

FIG. 2 shows a heating device being displaceable in the housing;

FIG. 3 shows a schematic partial sectional view in the area of a wick end in a near-wick position as the first setting position;

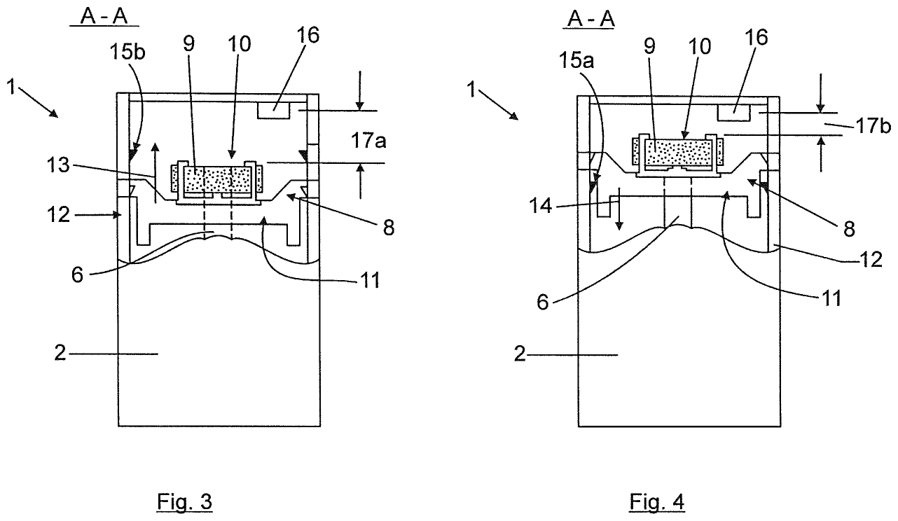

FIG. 4 shows the illustration of FIG. 3 in a remote-wick position corresponding to a second setting position;

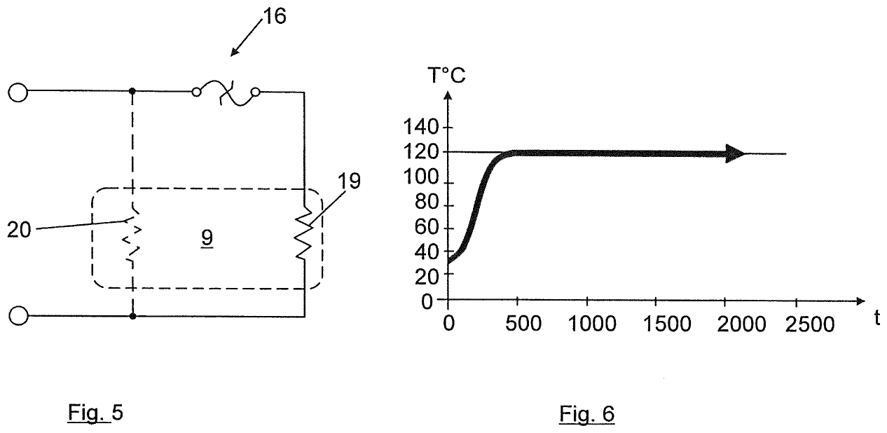

FIG. 5 shows a schematic circuit with a heating device in a near-wick position;

FIG. 6 shows a diagram with a temperature curve in the near-wick position;

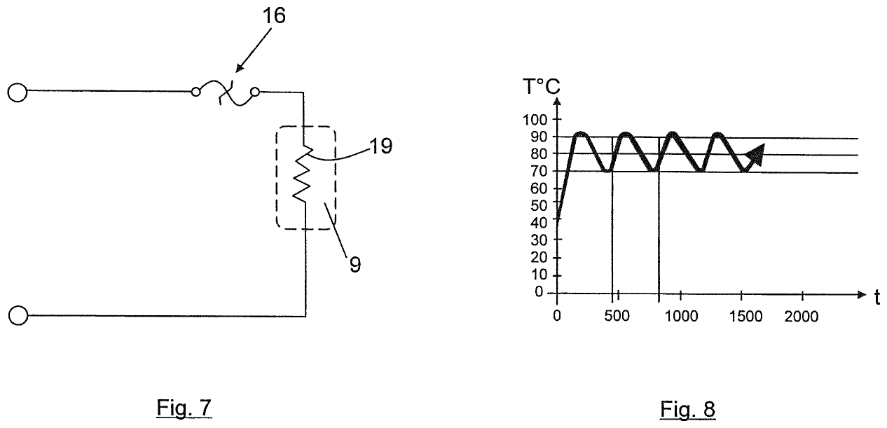

FIG. 7 shows a schematic circuit with a heating device in an embodiment with a heating resistor in a remote-wick position;

FIG. 8 shows a diagram showing a temperature curve of the arrangement according to FIG. 7;

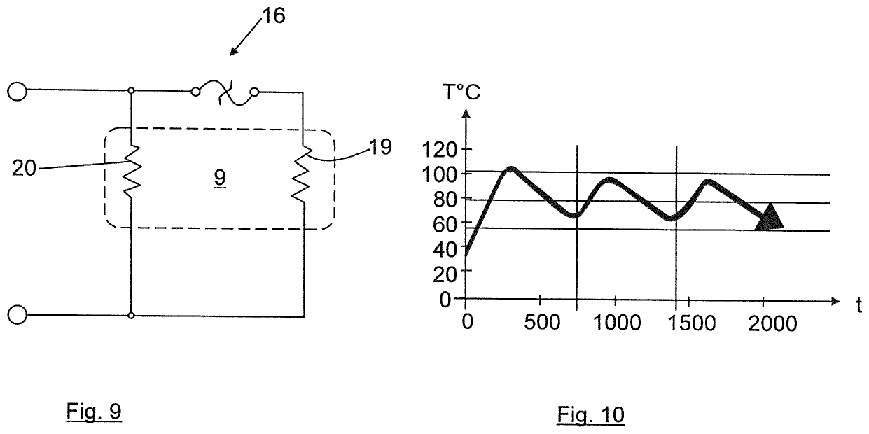

FIG. 9 shows a schematic circuit with a heating device in an embodiment with two heating resistors in a remote-wick position; and

FIG. 10 shows a diagram showing a temperature curve for an arrangement according to FIG. 9.

FIG. 1 shows a perspective view, to be understood merely as an example, of a device 1 for dispensing volatile substances, in particular fragrances and/or active substances, as a plug component. The device 1 comprises a housing 2 from which plug contacts 3 project, which can be plugged into an electrical socket (not shown) for the electrical power supply and for holding the housing 2 and which can be activated, for example, by a push-button 4.

A container 5 is inserted into the housing 2, here exemplarily from below, and is connected to the housing 2, preferably in an exchangeable and detachable manner. The container 5 contains the substance to be dispensed as well as a wick which projects upwards from the container 5 with a wick end 6 (see FIGS. 3, 4). Outlet openings 7 for the substance to be dispensed are formed in an upper wall of the container 5.

FIG. 2 shows as a separate component an exemplary electrical heating device 8 which has an exemplary cuboidal and annular heating body 9, e.g. made of a thermally conductive ceramic material, in which one or two heating resistors 19, 20 are embedded as examples for heating elements. The heating body 9 has a cylindrical opening, aligned in the direction of displacement of the heating device 8, as a wick-receiving opening 10, into which the wick end 6 can be inserted in a near-wick position as the first setting position according to FIG. 3. The heater 9 is displaceable on a vertically aligned sliding guide between an upper (second) and a lower (first) setting position, for which purpose the heating body is attached to a guide carriage 11 which is displaceable in vertical guide rails 12 of the housing 5.

In FIGS. 3 and 4, the heating device 8 with its heating body 9 is shown schematically in the housing 2 in the exclusively two possible setting positions shown here:

In FIG. 3, the heater 8 is shown in a lower position corresponding to a first setting position as a near-wick position in which the wick end is inserted into the wick-receiving opening 10 so that maximum heat transfer from the heating body 9 to the wick end 6 occurs during operation of the device 1.

In FIG. 4, on the other hand, the heating device 8 has been displaced respectively moved to an upper position corresponding to the second possible setting position, which corresponds to a remote-wick position in which the heating device 8 is spaced further away from the wick end 6 than in the near-wick position, so that the heating body 9, with reference to the vertical axis direction, lies at least with a partial region above the wick end 6 and a lower heat transfer to the wick end 6 takes place than in the near-wick position shown in FIG. 3.

In FIG. 3 the possible displacement upwards is indicated by the arrow 13 and in FIG. 4 the possible displacement downwards is indicated by the arrow 14, whereby in the two setting positions the heating device 8 is held on the housing side in each case by (schematically indicated) press-over detents 15a, 15b.

Above the heating device 8, as seen in the vertical axis direction, both in the near-wick position and in the remote-wick position, a thermoswitch 16, e.g. a bimetal switch, is arranged stationary in the housing 2 (here exemplarily in the housing 2 under the upper wall or ceiling wall). As a result, the distance 17a between the heating body 9 and the thermoswitch 16 in the near-wick position according to FIG. 3 is significantly greater than the distance 17b in the remote-wick position according to FIG. 4, so that the heat transfer to the thermoswitch 16 in the remote-wick position according to FIG. 3 is correspondingly significantly less than in the near-wick position according to FIG. 4.

As shown in FIG. 2, the plug contacts 3 and the thermoswitch 16 are stationary in the housing 2, so that the electrical supply lines 18 to the heating body 9 must be sufficiently long to allow and not hinder the linear displacement of the heater 8. In FIGS. 3 and 4, the electrical leads are not drawn for clarity.

FIG. 5 shows a highly schematised and merely exemplary heating circuit with a first heating resistor 19 and possibly with a second, parallel-connected heating resistor 20 (shown with dashed lines), which can be, as heating elements, part of the heating body 9 of the heating device 8. In this case, the thermoswitch 16 is connected upstream of the first heating resistor 19. The heating element 9 is located in the lower, large distance 17a away from the thermoswitch 16 in accordance with the near-wick position as shown in FIG. 3. Here, the heat transfer to the thermoswitch 16 is not sufficient to reach its switching temperature, e.g. 80° C., and to switch it from its closed position to its open position. Thus, heating by both heating resistors 19 and 20 is continuous, resulting in a wick temperature which, after an initial rise following the switching on of the device 1, is maintained at a constant level, here for example about 100° C.

FIG. 7 shows a circuit similar to FIG. 5, but with only one heating resistor 19 in the heating body 9 of the heating device 8. The thermoswitch 16 is connected upstream of the heating resistor here. The heating device 8 with the heating body 9 is here in the remote-wick position according to FIG. 4 and thus at a relatively small distance 17b from the thermoswitch 16. This results, for example, in a temperature curve of the wick temperature as shown in FIG. 8.

After the device 1 is switched on, the wick temperature and the temperature of the thermoswitch rise to, for example, about 80° C., whereby the thermoswitch is heated to its predefined or preset switching temperature and opens, resulting in the heating device 8 being switched off. Due to the stored heat in the heating body 9 and the temperature inertia of the arrangement, the wick temperature initially continues to rise in practice, for example to about 90° C. Only then does a cooling phase begin with a cooling of the wick temperature to, for example, about 70° C. Accordingly, the thermoswitch 16 has also reached its predefined or preset switching temperature again, whereby it is closed and the heating circuit also closes again, thereby reactivating the heating resistor and the wick temperature rises again. The wick temperature thus oscillates here in the manner of a sawtooth curve around an average temperature value of here exemplarily about 80° C. This avoids wick overheating with wick damage if the substance delivery rate is sufficiently high.

FIG. 9 shows a circuit similar to FIG. 7, which has a second heating resistor 20 connected in parallel with the first heating resistor 19, with the heating device 8 respectively the heating body 9 being also in the remote-wick position as shown in FIG. 4. This arrangement results in a wick temperature curve as shown in FIG. 10, again according to a sawtooth curve, but in comparison with FIG. 8 with larger temperature ranges and with longer cooling phases, since with a heating resistor 19 switched off by the thermoswitch 16 the parallel second heating element 20 continues to operate continuously. Depending on the circumstances, the oscillating temperature curve of the wick temperature can thus be designed and dimensioned differently.

Moreover, an operation corresponding to the alternating operation of the device 1 in FIGS. 7 and 9, in which the heating device is in a remote-wick position, can also be achieved in the near-wick position if the distance of the heating device 8 from the thermoswitch 16, the heating power of the heating device 8 and the switching temperature of the thermoswitch 16 are set up in such a way that even in the near-wick position of the heating device 8 the heat transfer to the thermoswitch 16 is sufficient to reach the switching temperature. As a result, when the switching temperature is reached, the thermal switch 16 opens to switch off the electric heating device 8 also in the near-wick position, which leads to an alternating operation of the device and its repeated switching on and off, but with the difference that the switch-on and switch-off cycles in the far-wick respectively remote-wick position are different, in particular shorter, than in the near-wick position.

### List of Reference Signs

1 Device
2 Housing
3 Plug contacts
4 Push-button
5 Container
6 Wick end
7 Outlet openings
8 Heating device
9 Heating body
10 Wick-receiving opening
11 Guide carriage
12 Guide rails
13 Arrow
14 Arrow
15a, b Detents
16 Thermoswitch
17a, b Distance
18 Supply lines
19 First heating resistor
20 Second heating resistor

1. Device for dispensing volatile substances,
with a housing (2),
with a container (5) for the substance to be dispensed,
with a wick received in the container (5) and protruding from the container (5) with a wick end (6) forming a substance dispensing area,
with an electrical heating device (8) which has an electrical heating circuit, the heating device (8) being displaceable relative to the wick end (6) in order to set a substance delivery rate,
characterized in that
the electrical heating circuit of the heating device (8) comprises a thermoswitch (16) being openable by heat exposure at a predetermined switching temperature and being arranged at a distance from the wick end (6), the thermoswitch (16) being switchable as a function of the distance between the heating device (8) and the thermoswitch (16).

2. Device according to claim 1, characterized in that
the heating device (8) is displaceable into a near-wick position and into a remote-wick position relative to the wick end (6) in order to set a substance delivery rate, with the heating device (8) being arranged closer to the wick end (6) in the near-wick position than in the remote-wick position and with a greater quantity of heat being transferred to the wick end (6) by the heating device (8), in its activated state, in the near-wick position than in the remote-wick position, and accordingly the substance delivery rate is greater in the near-wick position than in the remote-wick position,
the distance of the heating device (8) from the thermoswitch (16) is greater in the near-wick position than in the remote-wick position,
the thermoswitch (16) is switchable depending on the distance between the heating device (8) and the thermoswitch (16) in such way, and that the heating power of the heating device (8) and the switching temperature of the thermoswitch (16) are set in such way:
that, in the near-wick position of the heating device (8), the heat transfer to the thermoswitch (16) is too low to reach the switching temperature and accordingly the thermoswitch (16) closes or remains closed, and
that, in the remote-wick position of the heating device (8), the heat transfer to the thermoswitch (16) is sufficient to reach the switching temperature and, accordingly, the thermoswitch (16) opens to switch off the electrical heating device (8) when the switching temperature is reached, preferably in such a way that, in this remote-wick position of the heating device (8), the thermoswitch (16) repeatedly switches on and off for alternating operation of the device (1).

3. Device according to claim 1, characterized in that
the heating device (8) is displaceable into a near-wick position and into a remote-wick position relative to the wick end (6) in order to set a substance delivery rate, with the heating device (8) being arranged closer to the wick end (6) in the near-wick position than in the remote-wick position and with a greater quantity of heat being transferred to the wick end (6) by the heating device (8), in its activated state, in the near-wick position than in the remote-wick position, and accordingly the substance delivery rate is greater in the near-wick position than in the remote-wick position,
the distance of the heating device (8) from the thermoswitch (16) is greater in the near-wick position than in the remote-wick position,
the thermoswitch (16) is switchable depending on the distance between the heating device (8) and the thermoswitch (16) in such way, and that the heating power of the heating device (8) and the switching temperature of the thermoswitch (16) are set in such way:
that both in the remote-wick position of the heating device (8) and in the near-wick position of the heating device (8) the heat transfer to the thermoswitch (16) is sufficient to reach the switching temperature and accordingly the thermoswitch (16) opens upon reaching the switching temperature for switching off the heating device (8) such that the thermoswitch (16) repeatedly switches on and off both in the remote-wick position of the heating device (8) and in the near wick position of the heating device (8) for an alternating operation of the device (1), although with the difference that the switch-on and switch-off cycles in the remote-wick position are different, in particular shorter, than in the near wick position.

4. Device according to claim 2 or 3, characterized in that the heating device (8) is displaceable exclusively between the remote-wick position and the near-wick position and in this respect only two setting positions are provided.

5. Device according to any one of the preceding claims, characterized in that the thermoswitch (16) is arranged in the interior of the housing (19) and/or is arranged stationary.

6. Device according to any one of the preceding claims, characterized in that the thermoswitch (16) is a bimetal switch.

7. Apparatus according to any one of the preceding claims, characterized in that the heating device (8) comprises a single electrical heating element (19) being switchable with the thermoswitch (16).

8. Device according to any one of claims 1 to 6, characterized in that the heating device comprises two electrical heating elements (19, 20) arranged in parallel, with one heating element (19) being switchable with the thermoswitch (16).

9. Device according to any one of the preceding claims, characterized in that the heating device (8) comprises or is formed by a heating body (9), preferably plate-shaped and/or cuboidal and/or annular, with a wick-receiving opening (10) for the wick end (6).

10. Device according to any one of the preceding claims, characterized in that an electrical heating element (19, 20), which is a component of the electrical heating circuit, is embedded in the heating body (9), which is produced at least in regions, preferably predominantly, most preferably completely, from a thermally conductive material.

11. Device according to one of the preceding claims, characterized in that the heating device (8) is linearly displaceable on a sliding guide (12), preferably in such a way that the wick end (6) projects into the wick-receiving opening (10) in the near-wick position and in that, in the remote-wick position, the wick-receiving opening (10) lies at a distance above the wick end (6), with respect to the wick vertical axis direction, and accordingly has a smaller distance (17b) from the thermoswitch (16) than in the near-wick position.

12. Device according to one of the preceding claims, characterized in that the device (1) is designed as a plug component and has plug contacts (3) which project from the housing (2) and are pluggable into an electrical socket for supplying electrical power and for holding the housing (2).

13. A method of operating a device for dispensing volatile substances,
with a housing (2),
with a container (5) for the substance to be dispensed,
with a wick received in the container (5) and protruding from the container (5) with a wick end (6) forming a substance dispensing area,
with an electrical heating device (8) which has an electrical heating circuit, the heating device (8) being displaceable relative to the wick end (6) in order to set a substance delivery rate,
characterized in that
the electrical heating circuit of the heating device (8) comprises a thermoswitch (16) being openable by heat exposure at a predetermined switching temperature and being arranged at a distance from the wick end (6), the thermoswitch (16) being switched as a function of the distance between the heating device (8) and the thermoswitch (16).

14. A method according to claim 13, characterized in that
the heating device (8) is displaceable into a near-wick position and into a remote-wick position relative to the wick end (6) in order to set a substance delivery rate, with the heating device (8) being arranged closer to the wick end (6) in the near-wick position than in the remote-wick position and with a greater quantity of heat being transferred to the wick end (6) by the heating device (8), in its activated state, in the near-wick position than in the remote-wick position, and accordingly the substance delivery rate is greater in the near-wick position than in the remote-wick position,
the distance of the heating device (8) from the thermoswitch (16) is greater in the near-wick position than in the remote-wick position,
the thermoswitch (16) is switchable depending on the distance between the heating device (8) and the thermoswitch (16) in such way, and that the heating power of the heating device (8) and the switching temperature of the thermoswitch (16) are set in such way:
that, in the near-wick position of the heating device (8), the heat transfer to the thermoswitch (16) is too low to reach the switching temperature and, accordingly, the thermoswitch (16) closes or remains closed and the device (1) is operated continuously, and
that, in the wick-remote position of the heating device (8), the heat transfer to the thermoswitch (16) is sufficient to reach the switching temperature and, accordingly, the thermoswitch (16) opens to switch off the electrical heating device (8), preferably in such a way that, in this remote-wick position of the heating device (8), the thermoswitch (16) repeatedly switches on and off for alternating operation of the device (1).

15. A method according to claim 13, characterized in that
the heating device (8) is displaceable into a near-wick position and into a remote-wick position relative to the wick end (6) in order to set a substance delivery rate, with the heating device (8) being arranged closer to the wick end (6) in the near-wick position than in the remote-wick position and with a greater quantity of heat being transferred to the wick end (6) by the heating device (8), in its activated state, in the near-wick position than in the remote-wick position, and accordingly the substance delivery rate is greater in the near-wick position than in the remote-wick position,
the distance of the heating device (8) from the thermoswitch (16) is greater in the near-wick position than in the remote-wick position,
the thermoswitch (16) is switchable depending on the distance between the heating device (8) and the thermoswitch (16) in such way, and that the heating power of the heating device (8) and the switching temperature of the thermoswitch (16) are set in such way:
that both in the remote-wick position of the heating device (8) and in the near-wick position of the heating device (8) the heat transfer to the thermoswitch (16) is sufficient to reach the switching temperature and accordingly the thermoswitch (16) opens upon reaching the switching temperature for switching off the heating device (8) such that the thermoswitch (16) repeatedly switches on and off both in the remote-wick position of the heating device (8) and in the near wick position of the heating device (8) for an alternating operation of the device (1), although with the difference that the switch-on and switch-off cycles in the remote-wick position are different, in particular shorter, than in the near wick position.
